(19) United States
(12) Patent Application Publication
(10) Pub. No.: US 2021/0142873 A1
(43) Pub. Date: May 13, 2021

(54) METHOD FOR MONITORING INMATE'S HEALTH AND PROVIDING HEALTHCARE GUIDELINES

(71) Applicant: Spectrum Accountable Care Company, Las Vegas, NV (US)

(72) Inventors: Barry Goldstein, Las Vegas, NV (US); Quyen Tang Kiet, Huntington Beach, CA (US)

(73) Assignee: Spectrum Accountable Care Company

(21) Appl. No.: 16/929,701

(22) Filed: Jul. 15, 2020

Related U.S. Application Data
(60) Provisional application No. 62/934,815, filed on Nov. 13, 2019.

(51) Int. Cl. G16H 10/65; G16H 70/20; G16H 50/70; G06Q 50/22

(52) U.S. Cl. CPC G16H 10/65; G06Q 50/22; G16H 50/70; G16H 70/20

(57) ABSTRACT

A method monitors inmate's health and provides healthcare guidelines using the information gathered directly from each inmate and from information gathered from the general incarcerated inmate population in addition to the general medical information.

### RELATED APPLICATION

This application is a non-provisional of U.S. Provisional Pat. App. No. 62/934,815, filed on Nov. 13, 2019, the specification of which is included in its entirety by this reference.

### FIELD OF THE INVENTION

The present invention generally relates to illness diagnostic, and more specifically to a method for predicting illness occurring to an inmate population.

### BACKGROUND OF THE INVENTION

In the correctional healthcare space (correctional facilities like county jails, state/federal prisons, halfway houses, etc.) there is a need to quickly and accurately identify those confined individuals (“inmates”) who are at greatest risk for health-related problems in order to reduce liability due to negligent care. Further, because inmate health is constantly changing, often deteriorating the longer over the course of confinement, there is an ongoing need to identify such inmates in a timely manner and it is to this need that the present invention is primarily directed.

### SUMMARY OF THE INVENTION

The invention is directed to a process for monitoring inmate's health and providing healthcare guidelines using the information gathered directly from each inmate and from information gathered from the general incarcerated inmate population in addition to the general medical information.

The process can be divided into an immediate step performed at a holding jail when inmates are first taken into custody and a long term step performed at a prison where the inmates are sent.

In the immediate step, a determination is done based on the information provided by the inmates themselves, information collected from sensors of the inmate (temperature, body movement, breathing rate, etc.), and also the record that might be available from the arresting authority. The information gathered are fed into the system and the system determines if any specific and immediate action must be taken to avoid immediate health danger.

In the long term step, the health of the inmates is monitored regularly through periodic checkup and/or use of monitoring devices. The health information of each inmate is fed into the system and each physical condition is matched against a specific database. The results from different database are then combined to predict possible hidden physical problem and also to prescribe suitable treatment.

The health condition of each inmate is fed into the system and used for future diagnostic of other inmates, so the database and information in the system are continuously updated.

In one embodiment, the present invention provides a method for prescribing healthcare guidelines to an incarcerated inmate comprising receiving identification of an inmate, collecting health information from the inmate, prompting for additional health information based on the collected health information, storing the collected health information into an inmate database, comparing the collected health information for the inmate against a medical database, identifying a health condition based on a comparison result, and prescribing a treatment guideline according to the identified health condition.

In yet another embodiment, the present invention is an apparatus, for prescribing healthcare guidelines to an incarcerated inmate and comprises a data communication device for transmitting and receiving medical database to and from remote servers, a display device for displaying healthcare guidelines to a user, an IO controller for receiving health information of the inmate from the user, a storage unit for storing computer programs, the medical data and an inmate database, and a controller for executing the computer programs that causes the apparatus to receive inmate identification, collect the health information of the inmate, prompt for additional health information based on the collected health information, store the collected health information into the inmate database, compare the collected health information for the inmate against the medical database, identify a health condition based on a comparison result, and prescribe a treatment guideline according to the identified health condition.

The foregoing and other objects, features, aspects and advantages of the present invention will become better understood from a careful reading of a detailed description provided herein below with appropriate reference to the accompanying drawings.

### BRIEF DESCRIPTION OF THE DRAWINGS

The present invention can be understood in more detail by reading the subsequent detailed description in conjunction with the examples and references made to the accompanying drawings, wherein:

FIG. 1 is an illustration 100 of the system according to the present invention;

FIG. 2 is a process 200 describing the process for taking an inmate 102 into custody;

FIG. 3 illustrates an exemplary process 300 for continuously health monitoring of the inmates;

FIG. 4 illustrates a process 400 for improving the health database; and

FIG. 5 is an exemplary schematic diagram 500 of the server 106.

### DETAILED DESCRIPTION OF THE PREFERRED EMBODIMENTS

The present invention provides a system and a method that monitor the health of the inmates by continuously check their health conditions against known symptoms of diseases and prescribing guidelines for their health maintenance based a special prediction algorithm. The objective of the present invention is to apply artificial intelligence, deep learning, Bayesian predictive algorithms and/or software based heuristic automation to the an inmate's health information and other relevant data in order to identify inmates who are at greatest risk of acute health problems in the near term. In the long term CHICA further identifies, monitors and acquires information about all inmates in order to improve their long term and short term healthcare needs during their confinement.

In this application, Relevant Health Information (RHI) refers to any and all information that may assist in identifying and sorting an individual based on their health risks. For example, beyond the obvious measures of blood pressure, pulse and body temperature, RHI also includes age, body weight, body mass index, height, hair loss, skin color, crime, inmate associates, genetic information, social skill temperament (introversion versus extroversion), etc. RHI is not meant to be an exhaustive list but rather an open-ended definition that incorporates all possible individual information about an individual as such information will be part of the data set examined and processed by the artificial intelligence (or deep learning) software of the present system. RHI also includes the ongoing health condition of the inmate as a result of decisions made by the government agency during the course of the inmate's confinement.

Inmate in the context of the present application is an individual involuntarily and physically confined to the government's control such as an arrestee in a jail, prisoner in a correctional institution, or probationer in a halfway house. The status of being identified as an “inmate” for the purposes of this application begins when an individual is held involuntarily by a government agent, such as a policeman handcuffing a drunk driver—from this point, the driver is considered an “inmate” under this definition because said driver is now involuntarily confined to the control of the police officer.

The moment the government takes an individual into custody as an “inmate”, the government is fully responsible for the health and welfare of this inmate. If the inmate suffers any injury during the course of the inmate's confinement due to the government's failure to provide reasonable care, the government may be held accountable for the consequent financial damages for inmate injury suffered as a result of this failure. The system of the present application seeks to reduce the incidents of such failures by effectively identifying which inmates require closer healthcare attention.

In lieu of a fully staffed urgent care clinic, the system of the present invention seeks to apply artificial intelligence, deep learning, Bayesian predictive algorithms or any alternative heuristic automation that can quickly identify those inmates requiring immediate health monitoring. The system of the present invention engages in identification of RHI, acquisition of RHI, analysis of RHI, deep learning, and user report.
a. Identification of RHI—The inmate's relevant health information is whatever that is immediately available to the government agency confining the inmate. This can range from as little as nothing, which is often the case during a DUI arrest by the local police to a more thorough medical record often found in federal prisons, where inmates receive long term chronic care. The more RHI is identified the more accurate the health risk analysis; some RHI is always better than nothing.
b. Acquisition of RHI—The process of identifying, acquiring, storing and making the RHI available for analysis as a function of health risk is a critical component of the present system.
c. Analysis of RHI—After the RHI is made available for examination the system of the present invention employs a multitude of techniques, such as artificial intelligence, deep learning, risk analysis heuristic, and Bayesian predictive modeling, to study the RHI. The system defines, sorts, and identifies the inmate based on the risk parameters defined by the government agency or some other third-party standard.
d. Deep learning—The system of the present invention uses deep learning, also known as artificial intelligence modeling, and the data from the RHI analysis. The analysis of RHI improves over time because the datasets are constantly updated with corrected information based on previous analysis. For example, if based on a specifically defined set of data, an inmate's risk for health problems was previously ranked low and said inmate suffered a subsequent health incident, then the deep learning software would incorporate this new information and elevate the risk when similar dataset is observed in a future inmate.
e. User report—Once the analysis is completed and the inmate is properly ranked for risk based on previous data sets which improve over time as a function of updated information with corrections and reinforcement of previous analysis (“artificial intelligence learning model”), a report is provided to the government agency that prioritizes the inmates healthcare needs within the parameters defined by the government agency.

The RHI of an inmate may be gathered through observation when the inmate is taken into custody and the observable RHI includes alertness of the inmate, age, size, height, weight, skin pallor, skin complexion, etc. The RHI may also be acquired by placing a wearable device on the inmates to acquire RHI (pulse, temperature, blood pressure, etc.). After the RHI information is acquired, the information is entered, transferred, and converted to data (such as CSV format) loaded into a database on a server. This server may be networked and connected among unrelated government agencies all utilizing the same system. The RHI data on the server is used by all networked computers from different government agencies and the deep learning algorithm helps to improve the accuracy as the datasets are corrected and improved over time. The inmate's health condition is continuously monitored and the RHI is updated accordingly and shared among all the government agencies that employ the same system.

FIG. 1 is an illustration 100 of the system according to the present invention and FIG. 2 is a process 200 describing the process for taking an inmate 102 into custody. When an inmate 102 is taken into custody, the law enforcement officer 104 identifies the inmate 102, step 202, collects and records basic health related information, such as age, height, race, weight, etc., step 204. The officer 104 may inquire any known health condition or medicine that the inmate 102 is taken. The officer 104 may also collect the health information using different sensors and medical instruments. These sensors and medical instruments may collect the body temperature, the heart rate, the pulse, etc. As the health information is recorded, the system may suggest, in real time, follow up questions that the officer 104 may ask the inmate 102. The follow up questions are suggested by on the health information collected from the inmate 102. The information collected are stored in an inmate database on a server 106, which is connected to the Internet 108, step 206. As the information is entered, the system may prompt the office 104 to ask for additional input based on comparing the collected information with the stored health data in a medical database, step 208. The system may also provide some initial guidelines, step 212, for the care of the inmate 102 based on the health condition identified from the medical database, step 210. For example, if the inmate discloses that he is diabetic, the system will suggest the officer 104 to ask if the inmate 102 has his medicine with him or ask the inmate 102 to call his family to bring his medicine to the detention center.

After the inmate 102 is taken into custody 110, his health condition is continuously monitored by the staff 112 of the detention center. In special circumstances, special medical monitoring devices may be used by the inmates. These special monitoring devices may be a bracelet that measures the heart rate or a device that records steps taken each day by an inmate. The data collected by these wearable monitoring devices may be transmitted wirelessly to the server or downloaded periodically to the server. FIG. 3 illustrates an exemplary process 300 for continuously health monitoring of the inmates 102. The medical database (RHI) in the server 106 receives the latest medical information, step 302 and the medical database (health database) is updated continuously, step 304. After the health database is updated, the health database is compared with all the inmates in the inmate database, step 306. If any inmate's condition matches any new condition in the health database, step 308, a treatment guideline will be suggested, step 310. Using this system, an inmate previously considered healthy may be associated with a newly known medical condition.

The system of the present invention also improves the health database through the observational health data collected and input by the detention center staff. FIG. 4 illustrates a process 400 for improving the health database. If an inmate previously considered as being prone to disease AAA and develops symptom related to disease BBB, step 402, the association of the inmate's health condition with this newly developed symptom for disease BBB will be recorded, step 404 and the health database will be updated, step 408. This association may be unknown in the medical field because the unique environment condition the inmates face may cause the inmates more prone to some unique health problems not normally encountered by the general population. The updated health database will be used to check against the inmate database, step 410, to see if there is any inmate who may develop also symptom related to disease BBB. If an inmate is identified, step 412, a treatment will be recommended, step 414. This new association of the health conditions and the disease BBB will be shared with the medical professionals and the inmate populations from other detention facilities will also be checked against this newly found association.

The health diagnosis can be summarized as:

Diagnosis=F(medical health data from the medical field, inmate health data from the incarcerated population, inmate's health condition)

The medical health database contains the RHI collected from the database commonly available to the medical professionals. The inmate health data may be collected from other detention centers or the prisons.

FIG. 5 is an exemplary schematic diagram 500 of the server 106. The server 106 comprises a display device 502 for interfacing with users, a storage device 504 for storing health database and the computer program that implements the present invention, an IO controller 506 for interfacing with the users, a data communication device 508 for communication with the Internet or other servers, and a CPU 510 for implementing the process and the method of the present invention. The server 106 may be implemented differently, including implemented on a single chip.

In operation, the system of the present invention assists a law enforcement officer to take health related information from an inmate by recording the health information provided by the inmate and suggesting follow up questions to be asked to the inmate. The information collected are stored in the inmate's file and also into the general medical database and the inmate health database. The inmate's physical condition is monitored constantly and the health information is updated continuously. If the inmate's health condition matches symptoms of any known disease, the system will generate a recommended instruction for the care of this inmate. If the inmate becomes ill even the health conditions do not match any known disease, a new association between the inmate's health condition and the symptoms will be made and this new information will be incorporated in the inmate health database for use on other inmates.

Although the present invention has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims. It is understood that features shown in different figures and described in different embodiments can be easily combined within the scope of the invention. The processes described in this specification may be implemented by the above disclosed hardware and may also be implemented by stored software code executed by the CPU.

Modifications, additions, or omissions may be made to the systems and methods described without departing from the scope of the disclosure. The components of the systems and methods described may be integrated or separated according to particular needs. Moreover, the operations of the systems and methods described may be performed by more, fewer, or other components without departing from the scope of the present disclosure.

Although the present disclosure has been described with several embodiments, sundry changes, substitutions, variations, alterations, and modifications can be suggested to one skilled in the art, and it is intended that the disclosure encompass all such changes, substitutions, variations, alterations, and modifications falling within the spirit and scope of the appended claims.

What is claimed is:

1. A method for prescribing healthcare guidelines to an incarcerated inmate comprising:
receiving identification of an inmate;
collecting health information from the inmate;
prompting for additional health information based on the collected health information;
storing the collected health information into an inmate database;
comparing the collected health information for the inmate against a medical database;
identifying a health condition based on a comparison result; and
prescribing a treatment guideline according to the identified health condition.

2. The method of claim 1, further comprising:
receiving a new health condition on the inmate;
associating the new health condition with the collected health information for the inmate; and
recording the association of the new health condition with the collected health information for the inmate onto the medical database.

3. The method of claim 2, further comprising checking the collected health information of other inmates listed in the inmate database against the medical database.

4. The method of claim 2, further comprising transmitting the association of the new health condition with the collected health information for the inmate to other medical databases.

5. The method of claim 1, wherein the medical database is updated with medical information from third parties and with the collected health information stored in the inmate database.

6. The method of claim 1, further comprising:
communicating with wearable health data collector placed onto the inmate; and
receiving health information transmitted by wearable health data collector.

7. An apparatus, for prescribing healthcare guidelines to an incarcerated inmate, comprising:
a data communication device for transmitting and receiving medical database to and from remote servers;
a display device for displaying healthcare guidelines to a user;
an IO controller for receiving health information of the inmate from the user;
a storage unit for storing computer programs, the medical data and an inmate database; and
a controller for executing the computer programs that causes the apparatus to
receive inmate identification,
collect the health information of the inmate,
prompt for additional health information based on the collected health information,
store the collected health information into the inmate database,
compare the collected health information for the inmate against the medical database,
identify a health condition based on a comparison result, and
prescribe a treatment guideline according to the identified health condition.

8. The apparatus of claim 7, wherein the computer executes the computer programs further causes the apparatus to
receive a new health condition on the inmate,
associate the new health condition with the collected health information for the inmate, and
record the association of the new health condition with the collected health information for the inmate onto the medical database.

9. The apparatus of claim 7, wherein the computer executes the computer programs further causes the apparatus to check the collected health information of other inmates listed in the inmate database against the medical database.

10. The apparatus of claim 7, wherein the computer executes the computer programs further causes the apparatus to transmit the association of the new health condition with the collected health information for the inmate to other medical databases.

11. The apparatus of claim 7, wherein the computer executes the computer programs further causes the apparatus to communicate with wearable health data collector placed onto the inmate, and receive health information transmitted by wearable health data collector.

12. The apparatus of claim 7, wherein the medical database is updated with medical information from third parties and with the collected health information stored in the inmate database.
